(19) United States
(12) Patent Application Publication
(10) Pub. No.: US 2012/0215107 A1
(43) Pub. Date: Aug. 23, 2012

(54) ULTRASOUND PROBE AND ULTRASOUND DIAGNOSTIC APPARATUS

(75) Inventor: Katsuya YAMAMOTO, Ashigara-kami-gun (JP)

(73) Assignee: FUJIFILM CORPORATION, Tokyo (JP)

(21) Appl. No.: 13/362,521

(22) Filed: Jan. 31, 2012

(30) Foreign Application Priority Data
Feb. 17, 2011 (JP) 2011-031805

(51) Int. Cl. A61B 8/14

(52) U.S. Cl. 600/441

(57) ABSTRACT

An ultrasound probe includes a first transducer array for a B-mode image which transmits an ultrasonic beam for a B-mode image, a second transducer array for sound speed measurement which is tiltably provided and transmits an ultrasonic beam for sound speed measurement, and a tilting unit which tilts the second transducer array for sound speed measurement in accordance with the angle of an abdominal wall of the subject.

### BACKGROUND OF THE INVENTION

The present invention relates to an ultrasound probe and an ultrasound diagnostic apparatus, and in particular, to an ultrasound probe and an ultrasound diagnostic apparatus for performing both production of a B-mode image and measurement of sound speed.

An ultrasound diagnostic apparatus using an ultrasound image has hitherto been put into practical use in the field of medicine. In general, this type of ultrasound diagnostic apparatus has an ultrasound probe equipped with a transducer array and an apparatus body connected to the ultrasound probe. An ultrasonic beam is transmitted from the ultrasound probe toward a subject, an ultrasonic echo from the subject is received by the ultrasound probe, and the reception signal is electrically processed by the apparatus body to produce an ultrasound image.

In recent years, in order to diagnose a region under diagnosis in the subject with satisfactory precision, a sound speed in the region under diagnosis is measured.

For example, JP 2010-99452 A describes an ultrasound diagnostic apparatus which sets a plurality of lattice points in the vicinity of the region under diagnosis, and a local sound speed value is calculated on the basis of reception data obtained by transmitting and receiving an ultrasonic beam for each lattice point.

In the apparatus described in JP 2010-99452 A, the ultrasonic beam is transmitted and received from the ultrasound probe toward the subject to calculate the local sound speed value in the region under diagnosis, making it possible to display information of the local sound speed on the B-mode image in an overlapping manner. If a sound speed map which represents the distribution of the local sound speed values at a plurality of points in a predetermine region is produced and displayed along with a B-mode image, it is effective to diagnose the region under diagnosis.

However, near an abdominal wall which covers the internal organs or the like of the subject has sound speed different from other parts due to the presence of fat or the like. For this reason, there is a problem in that it is difficult to accurately measure sound speed since, when an ultrasonic beam transmitted from the ultrasound probe passes through the abdominal wall, the ultrasonic beam is refracted depending on the entrance angle to the abdominal wall.

### SUMMARY OF THE INVENTION

The invention has been finalized in order to solve the problems in the related art, and an object of the invention is to provide an ultrasound probe and an ultrasound diagnostic apparatus capable of reducing the influence of ultrasonic beam refraction from an abdominal wall to perform B-mode image production and accurate sound speed measurement.

An ultrasound probe according to the present invention includes a first transducer array for a B-mode image which transmits an ultrasonic beam for a B-mode image, a second transducer array for sound speed measurement which is tiltably provided and transmits an ultrasonic beam for sound speed measurement, and a tilting unit which tilts the second transducer array for sound speed measurement in accordance with the angle of an abdominal wall of the subject.

An ultrasound diagnostic apparatus according to the present invention includes the above-described ultrasound probe, an image producer which produces a B-mode image on the basis of reception data for a B-mode image obtained by the reception circuit, an abdominal wall detector which detects the angle of an abdominal wall of a subject on the B-mode image produced by the image producer, a controller which adjusts the tilt angle of the transducer array for sound speed measurement by the tilting unit in accordance with the angle of the abdominal wall detected by the abdominal wall detector, and a sound speed map producer which produces a sound speed map on the basis of reception data for sound speed measurement obtained by the reception circuit.

### BRIEF DESCRIPTION OF THE DRAWINGS

FIG. 1 is a block diagram showing the configuration of an ultrasound diagnostic apparatus including an ultrasound probe according to Embodiment 1 of the invention.

FIG. 2 is a front view schematically showing a transducer array for a B-mode image and a transducer array for sound speed measurement of an ultrasound probe according to Embodiment 1.

FIG. 3 is a diagram schematically showing a B-mode image.

FIG. 4 is a diagram schematically showing the principle of sound speed calculation in Embodiment 1.

FIGS. 5A and 5B are a front view and a side view schematically showing a transducer array for a B-mode image and a transducer array for sound speed measurement of an ultrasound probe according to a modification of Embodiment 1.

FIG. 6 is a block diagram showing the configuration of an ultrasound probe according to Embodiment 2.

FIG. 7 is a block diagram showing the configuration of an ultrasound probe according to Embodiment 3.

FIGS. 8A and 8B are a front view and a side view schematically showing a transducer array for a B-mode image and a transducer array for sound speed measurement of an ultrasound probe according to Embodiment 3.

FIGS. 9A and 9B are a front view and a side view schematically showing a transducer array for a B-mode image and a transducer array for sound speed measurement of an ultrasound probe according to a modification of Embodiment 3.

### DETAILED DESCRIPTION OF THE INVENTION

Hereinafter, embodiments of the invention will be described with reference to the accompanying drawings.

### Embodiment 1

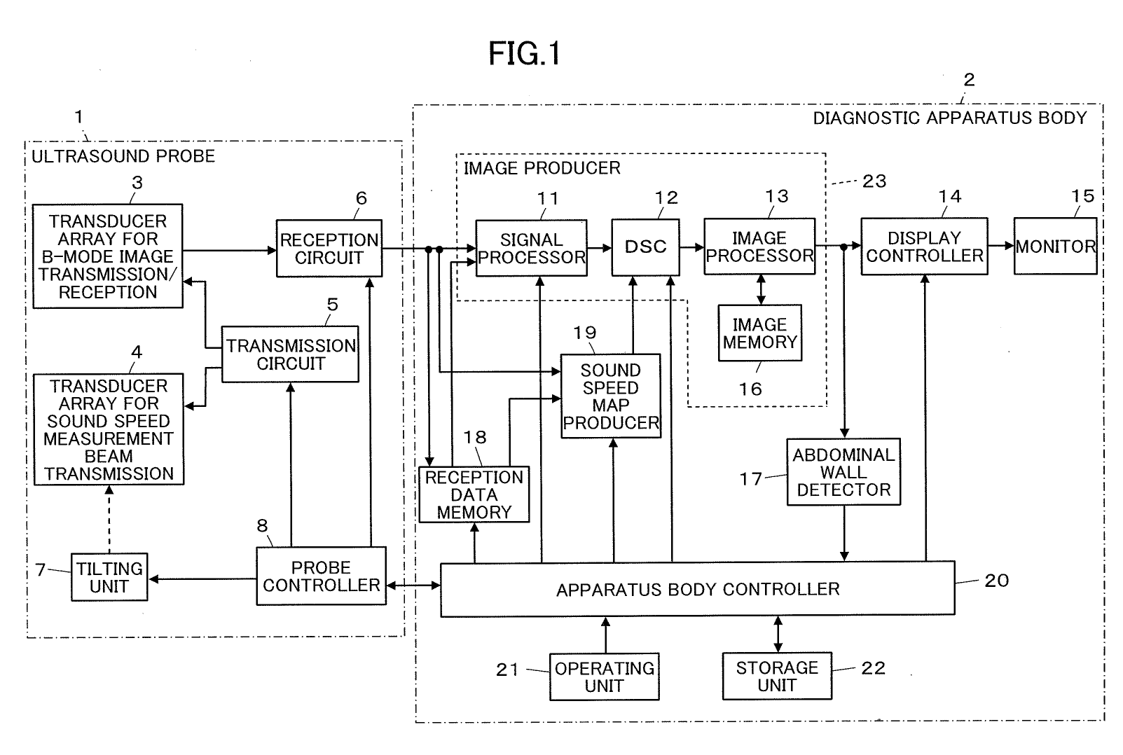

FIG. 1 shows the configuration of an ultrasound diagnostic apparatus including an ultrasound probe 1 according to Embodiment 1 of the invention. A diagnostic apparatus body 2 is connected to the ultrasound probe 1.

An ultrasound probe 1 includes a transducer array 3 for B-mode image transmission/reception and a transducer array 4 for sound speed measurement beam transmission. A transmission circuit 5 is connected to the transducer array 3 for B-mode image transmission/reception and the transducer array 4 for sound speed measurement beam transmission. A reception circuit 6 is connected to the transducer array 3 for B-mode image transmission/reception.

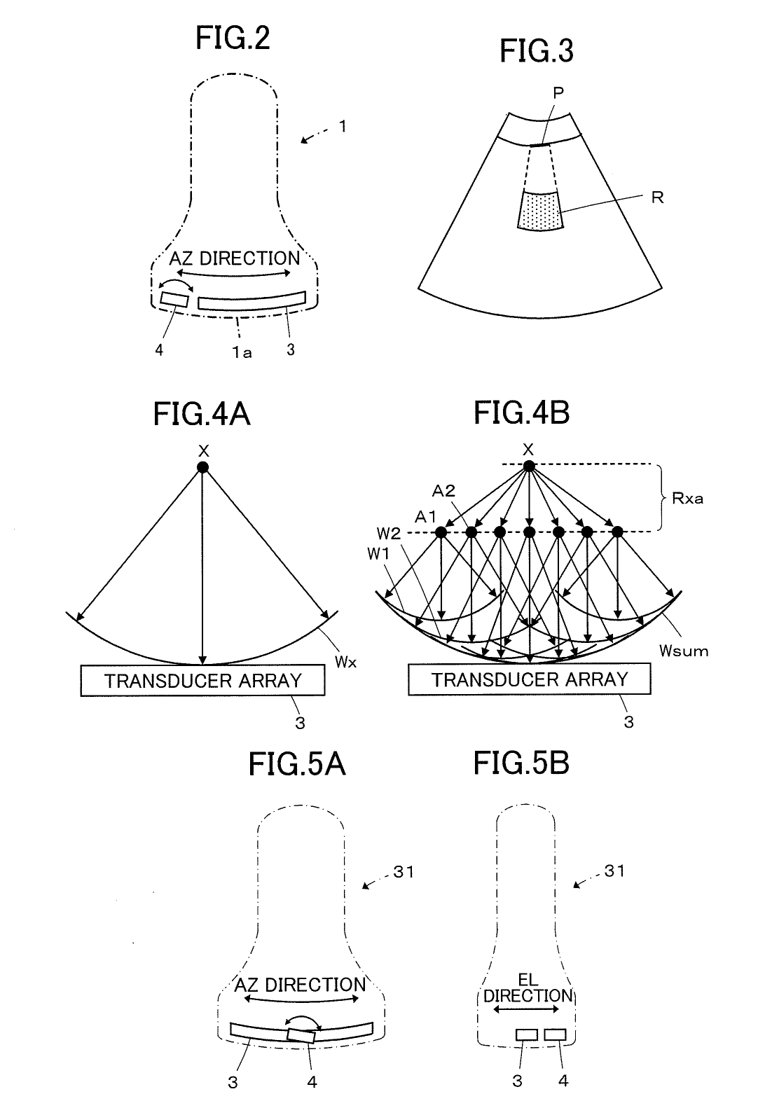

As shown in FIG. 2, the transducer array 3 for B-mode image transmission/reception and the transducer array 4 for sound speed measurement beam transmission are disposed such that their azimuth directions are identical to each other in the vicinity of a subject abutment portion 1a of the ultrasound probe 1. The transducer array 4 for sound speed measurement beam transmission is located adjacently in the azimuth direction of the transducer array 3 for B-mode image transmission/reception. The transducer array 3 for B-mode image transmission/reception is fixed in the ultrasound probe 1, while the transducer array 4 for sound speed measurement beam transmission is provided to be tiltable in its azimuth direction in the ultrasound probe 1. A tilting unit 7 for tilting the transducer array 4 for sound speed measurement beam transmission is connected to the transducer array 4 for sound speed measurement beam transmission. A probe controller 8 is connected to the transmission circuit 5, the reception circuit 6, and the tilting unit 7.

The diagnostic apparatus body 2 has a signal processor 11 connected to the reception circuit 6 of the ultrasound probe 1, and a DSC (Digital Scan Converter) 12, an image processor 13, a display controller 14, and a monitor 15 are sequentially connected to the signal processor 11. An image memory 16 and an abdominal wall detector 17 are connected to the image processor 13. The diagnostic apparatus body 2 has a reception data memory 18 and a sound speed map producer 19 each of which are connected to the reception circuit 6 of the ultrasound probe 1. An apparatus body controller 20 is connected to the signal processor 11, the DSC 12, the display controller 14, the abdominal wall detector 17, the reception data memory 18, and the sound speed map producer 19. An operating unit 21 and a storage unit 22 are connected to the apparatus body controller 20.

The probe controller 8 of the ultrasound probe 1 and the apparatus body controller 20 of the diagnostic apparatus body 2 are connected to each other.

Each of the transducer array 3 for B-mode image transmission/reception and the transducer array 4 for sound speed measurement beam transmission has a plurality of ultrasound transducers arranged in a one-dimensional or two-dimensional manner. The ultrasound transducers of the transducer array 3 for B-mode image transmission/reception transmit ultrasonic waves for B-mode image production in response to driving signals supplied from the transmission circuit 5. The ultrasound transducers of the transducer array 4 for sound speed measurement beam transmission transmit ultrasonic waves for sound speed measurement in response to driving signals supplied from the transmission circuit 5. Each of the ultrasound transducers of the transducer array 3 for B-mode image transmission/reception receives an ultrasonic echo of the ultrasonic wave for B-mode image production from the subject and outputs a reception signal, and receives an ultrasonic echo of the ultrasonic wave for sound speed measurement from the subject and outputs a reception signal. That is, the transducer array 3 for B-mode image transmission/reception receives ultrasonic echo for both B-mode image production and sound speed measurement.

Each ultrasound transducer of the transducer array 3 for B-mode image transmission/reception and the transducer array 4 for sound speed measurement is constituted by a vibrator in which electrodes are formed at both ends of a piezoelectric body made of piezoelectric ceramic represented by PZT (Pb (lead) zirconate titanate), a polymer piezoelectric device represented by PVDF (polyvinylidene difluoride), piezoelectric single crystal represented by PMN-PT (lead magnesium niobate-lead titanate solid solution), or the like.

If a pulsed or continuous-wave voltage is applied across the electrodes of the vibrator, the piezoelectric body expands and contracts, thereby pulsed or continuous-wave ultrasonic waves are produced from the vibrators and synthesized to form an ultrasonic beam. When receiving the propagating ultrasonic waves, the vibrators expand and contract to produce electric signals, and the electric signals are output as the reception signals of the ultrasonic waves.

The transmission circuit 5 includes, for example, a plurality of pulsars. The transmission circuit 5 adjusts the delay amount of each of the driving signals on the basis of a transmission delay pattern selected in response to a control signal from the probe controller 8 such that ultrasonic waves transmitted from the ultrasound transducers of the transducer array 3 for B-mode image transmission/reception form an ultrasonic beam, and supplies the adjusted driving signals to the ultrasound transducers of the transducer array 3. Simultaneously, the transmission circuit 5 adjusts the delay amount of each of the driving signals such that ultrasonic waves transmitted from the ultrasound transducers of the transducer array 4 for sound speed measurement beam transmission are focused on a point where sound speed should be measured, and supplies the adjusted driving signals to the ultrasound transducers of the transducer array 4.

The reception circuit 6 amplifies the reception signals output from the ultrasound transducers of the transducer array 3 for B-mode image transmission/reception, and performs A/D conversion for the amplified reception signals. The reception circuit 6, thereafter, performs a reception focus process by giving a delay to each of the reception signals in accordance with the sound speed or the distribution of sound speed set on the basis of a reception delay pattern selected in response to a control signal from the probe controller 8, and adding the reception signals. With this reception focus process, the focus of the ultrasonic echo is narrowed to produce reception data (sound ray signal).

The tilting unit 7 is used to tilt the transducer array 4 for sound speed measurement beam transmission in response to a control signal from the probe controller 8, and various actuators using an electric motor, an electric cylinder, or the like may be used.

The probe controller 8 controls the respective units of the ultrasound probe 1 on the basis of various control signals transmitted from the apparatus body controller 20 of the diagnostic apparatus body 2.

The signal processor 11 of the diagnostic apparatus body 2 corrects attenuation depending on the distance in accordance with the depth of the reflection position of the ultrasonic wave for reception data produced by the reception circuit 6 of the ultrasound probe 1, and performs an envelope detection process to produce a B-mode image signal which is tomographic image information relating to the tissue of the subject.

The DSC 12 converts (raster-converts) the B-mode image signal produced by the signal processor 11 to an image signal based on a normal television signal scan system.

The image processor 13 performs various necessary image processes, such as a gradation process, on the B-mode image signal input from the DSC 12, and outputs the processed B-mode image signal to the display controller 14 or stores the processed B-mode image signal in the image memory 16.

The signal processor 11, the DSC 12, the image processor 13, and the image memory 16 form an image producer 23.

The display controller 14 displays an ultrasound diagnostic image on the monitor 15 on the basis of the B-mode image signal subjected to the image process by the image processor 13.

The monitor 15 includes, for example, a display device, such as an LCD, and displays the ultrasound diagnostic image under the control of the display controller 14.

As shown in FIG. 3, the abdominal wall detector 17 detects an abdominal wall P of the subject above a region of interest R set in the B-mode image and detects the angle of the abdominal wall P on the basis of the B-mode image signal subjected to the image process by the image processor 13.

The reception data memory 18 sequentially stores reception data output from the reception circuit 6. The reception data memory 18 also stores information (for example, parameters representing the depth of the reflection position of the ultrasonic wave, the density of the scan lines, and the width of the field of vision) relating to a frame rate input from the apparatus body controller 20 in association with reception data.

The sound speed map producer 19 calculates a local sound speed value in the tissue of the subject as a diagnosis target on the basis of reception data stored in the reception data memory 18 under the control of the apparatus body controller 20, and produces a sound speed map.

The apparatus body controller 20 controls the respective units of the ultrasound diagnostic apparatus on the basis of a command input from the operating unit 21 by the operator.

The operating unit 21 is used when the operator performs an input operation, and may be constituted by a keyboard, a mouse, a trackball, a touch panel, or the like.

The storage unit 22 stores an operation program or the like, and may be constituted by, for example, a recording medium such as an MO, an MT, a RAM, a CD-ROM, a DVD-ROM, an SD card, a CF card, or a USB memory, or a server.

The signal processor 11, the DSC 12, the image processor 13, the display controller 14, and the sound speed map producer 19 are constituted by a CPU and an operation program which causes the CPU to perform various processes, and these may be constituted by digital circuits.

The operator can select one of the following three display modes by using the operating unit 21. That is, display can be performed in a desired mode from among a mode in which a B-mode image is displayed alone, a mode in which a sound speed map is displayed on a B-mode image in an overlapping manner (for example, display where color or luminance changes depending on a local sound speed value or display where points having the same local sound speed value are connected by a line), and a mode in which a B-mode image and a sound speed map image are displayed in parallel.

When displaying a B-mode image, first, ultrasonic waves are transmitted from the ultrasound transducers of the transducer array 3 for B-mode image transmission/reception in response to the driving signals from the transmission circuit 5 of the ultrasound probe 1, the reception signal from each ultrasound transducer having received the ultrasonic echo from the subject is output to the reception circuit 6, and reception data is produced by the reception circuit 6. A B-mode image signal is produced by the signal processor 11 of the diagnostic apparatus body 2 to which reception data is input and is then raster-converted by the DSC 12, and various image processes are performed on the B-mode image signal in the image processor 13. Thereafter, an ultrasound diagnostic image is displayed on the monitor 15 on the basis of the B-mode image signal by the display controller 14.

The calculation of the local sound speed value may be performed by the method described in JP 2010-99452 A filed in the name of the applicant.

As shown in FIG. 4A, according to this method, when the ultrasonic waves are transmitted into the subject, a received wave Wx reaches the transducer array 3 from a lattice point X as a reflection point of the subject. Then, as shown in FIG. 4B, a plurality of lattice points A1, A2, . . . are arranged at regular intervals at positions shallower than the lattice point X, that is, at positions closer to the transducer array 3. Then, the local sonic speed at the lattice point X is obtained according to the Huygens principle whereby a synthesized wave Wsum of received waves W1, W2, . . . from a plurality of lattice points A1, A2, . . . having received the received wave from the lattice point X coincides with the received wave Wx from the lattice point X.

First, the optimum sound speed values for all the lattice points X, A1, A2, . . . are obtained. The optimum sound speed value is a speed sound value such that imaging is performed with focus calculation based on the set speed sound for each lattice point to form an ultrasound image, and when the set sound speed changes in various ways, contrast and sharpness of the image become highest. For example, as described in JP 8-317926 A, the optimum sound speed value can be determined on the basis of contrast of an image, a spatial frequency in a scan direction, dispersion, or the like.

Next, the waveform of a virtual received wave Wx emitted from the lattice point X is calculated using the optimum sound speed value for the lattice point X.

A virtual local sound speed value V at the lattice point X changes in various ways to calculate a virtual synthesized wave Wsum of the received waves W1, W2, . . . from the lattice points A1, A2, . . . . At this time, it is assumed that the sound speed is uniform in a region Rxa between the lattice point X and each of the lattice points A1, A2, . . . , and is equal to the local sound speed value V at the lattice point X. The time until the ultrasonic wave propagating from the lattice point X reaches the lattice points A1, A2, . . . becomes XA1/V, XA2/V, . . . , where XA1, XA2, . . . designate the distance between the respective lattice points A1, A2, . . . and the lattice point X, respectively. Accordingly, reflected waves emitted from the lattice points A1, A2, . . . with the delay of the time XA1/V, XA2/V, . . . are synthesized, thereby obtaining the virtual synthesized wave Wsum.

Next, deviations between a plurality of virtual synthesized waves Wsum calculated by changing the virtual local sound speed value V at the lattice point X and the virtual received wave Wx from the lattice point X are calculated, and the virtual local sound speed value V with the minimum deviation is determined to be the local sound speed value at the lattice point X. As the method of calculating the deviation between the virtual synthesized wave Wsum and the virtual received wave Wx from the lattice point X, a method in which an intercorrelation is made, a method in which phase matching addition is performed while the delay obtained from the synthesized wave Wsum is applied to the received wave Wx, a method in which phase matching addition is performed while the delay obtained from the received Wx is applied to the synthesized wave Wsum, or the like may be used.

In the above-described manner, it is possible to calculate the local speed sound value in the subject on the basis of reception data produced by the reception circuit 6 of the ultrasound probe 1 with high precision. Similarly, it is possible to produce the sound speed map which represents the distribution of the local sound speed values in the set region of interest.

Next, the operation of Embodiment 1 will be described.

First, an ultrasonic beam for a B-mode image is transmitted from the plurality of ultrasound transducers of the transducer array 3 for B-mode image transmission/reception in response to the driving signals from the transmission circuit 5 of the ultrasound probe 1, and a reception signal from each ultrasound transducer having received an ultrasonic echo from the subject is output to the reception circuit 6 to produce reception data for a B-mode image. A B-mode image is displayed on the monitor 15 by the display controller 14 on the basis of the B-mode image signal produced by the image producer 23 of the diagnostic apparatus body 2.

If the operator operates the operating unit 21 to set a region of interest R on the B-mode image displayed on the monitor 15, a plurality of lattice points are set in the region of interest R by the apparatus body controller 20. As shown in FIG. 3, the angle of the abdominal wall P of the subject above the region of interest R is detected by the abdominal wall detector 17. The detected angle of the abdominal wall P is transmitted from the apparatus body controller 20 of the diagnostic apparatus body 2 to the probe controller 8 of the ultrasound probe 1, and the tilting unit 7 is actuated by the probe controller 8 such that the tilt angle of the transducer array 4 for sound speed measurement beam transmission is substantially the same as the angle of the abdominal wall P.

Next, the transmission circuit 5 and the reception circuit 6 are controlled by the probe controller 8, and transmission and reception of ultrasonic beams for sound speed measurement are sequentially performed while forming a transmission focus at each of the plurality of lattice points set in the region of interest R. At this time, the driving signals from the transmission circuit 5 are supplied to the transducer array 4 for sound speed measurement beam transmission, and an ultrasonic beam is transmitted from the plurality of ultrasound transducers of the transducer array 4 for sound speed measurement beam transmission. Meanwhile, the transducer array 4 for sound speed measurement beam transmission is adjusted to the tilt angle which is substantially the same as the angle of the abdominal wall P above the region of interest R. For this reason, the ultrasonic beam transmitted from the transducer array 4 for sound speed measurement beam transmission substantially enters the abdominal wall P vertically, passes through the abdominal wall P to form a transmission focus at each lattice point with little influence of refraction from the abdominal wall P. An ultrasonic echo from the subject is received by the plurality of ultrasound transducers of the transducer array 3 for B-mode image transmission/reception.

Each time an ultrasonic beam is received in such a manner, reception data for sound speed measurement produced by the reception circuit 6 is sequentially stored in the reception data memory 18. If reception data for sound speed measurement for all the lattice points in the region of interest R are acquired, a command to form a sound speed map is output from the apparatus body controller 20 to the sound speed map producer 19. The sound speed map producer 19 calculates the local sound speed value at each lattice point using reception data for sound speed measurement from among reception data stored in the reception data memory 18 to produce the sound speed map in the region of interest R. Data relating to the sound speed map obtained by the sound speed map producer 19 is raster-converted by the DSC 12, is subjected to various image processes by the image processor 13, and is then sent to the display controller 14. The B-mode image and the sound speed map are displayed on the monitor 15 in an overlapping manner or the B-mode image and the sound speed map image are displayed on the monitor 15 in parallel in accordance with the display mode input from the operating unit 21 by the operator.

Reception data for sound speed measurement produced by the reception circuit 6 is stored in the reception data memory 18 and also input to the signal processor 11 of the image producer 23. At this time, the actuation of the signal processor 11 is stopped in accordance with a command from the apparatus body controller 20, thereby preventing the B-mode image signal from being produced using reception data for sound speed measurement.

As described above, the tilting unit 7 is used to adjust the tilt angle of the transducer array 4 for sound speed measurement beam transmission to be the same as the angle of the abdominal wall P of the subject above the region of interest R, making it possible to reduce the influence of refraction of the ultrasonic beam from the abdominal wall P and to perform accurate sound speed measurement.

The number of channels of the transducer array 3 for B-mode image transmission/reception, the number of channels of the transducer array 4 for sound speed measurement beam transmission, the center frequency of an ultrasonic beam for a B-mode image, and the center frequency of an ultrasonic beam for sound speed measurement can be appropriately selected.

Although in Embodiment 1, as shown in FIG. 2, the transducer array 4 for sound speed measurement beam transmission is disposed adjacently in the azimuth direction of the transducer array 3 for B-mode image transmission/reception, the invention is not limited thereto. Like an ultrasound probe 31 shown in FIGS. 5A and 5B, a configuration may be made in which a transducer array 4 for sound speed measurement beam transmission disposed adjacently in the elevation direction of the transducer array 3 for B-mode image transmission/reception is tilted in the azimuth direction.

### Embodiment 2

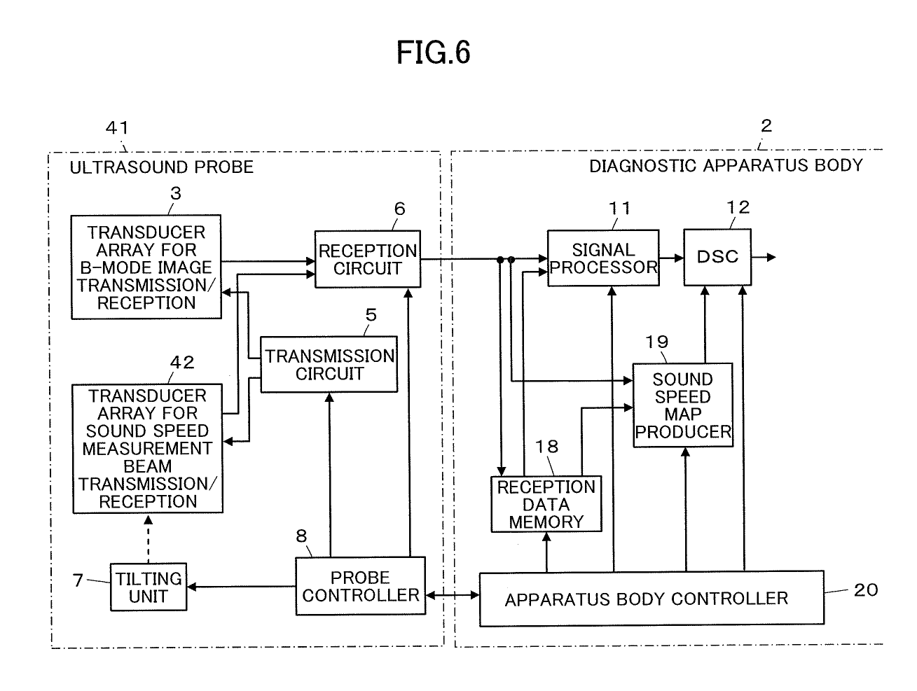

FIG. 6 shows the configuration of an ultrasound probe 41 according to Embodiment 2. The ultrasound probe 41 includes a transducer array 42 for sound speed measurement beam transmission/reception, instead of the transducer array 4 for sound speed measurement beam transmission in the ultrasound probe 1 of Embodiment 1 shown in FIG. 1. The transmission circuit 5, the reception circuit 6, and the tilting unit 7 are connected to the transducer array 42 for sound speed measurement beam transmission/reception.

Similarly to the transducer array 4 for sound speed measurement beam transmission of Embodiment 1, the transducer array 42 for sound speed measurement beam transmission/reception sequentially transmits ultrasonic beams for sound speed measurement while forming a transmission focus at a plurality of lattice points in the region of interest R on the basis of the driving signals supplied from the transmission circuit 5. The transducer array 42 for sound speed measurement beam transmission/reception also receives an ultrasonic echo corresponding to each transmitted ultrasonic beam and outputs reception signals to the reception circuit 6.

As described above, even when transmission and reception of ultrasonic beams for sound speed measurement are performed using the transducer array 42 for sound speed measurement beam transmission/reception, as in Embodiment 1, the tilt angle of the transducer array 42 for sound speed measurement beam transmission/reception is adjusted by the tilting unit 7, thereby reducing the influence of refraction of an ultrasonic beam from the abdominal wall P to perform accurate sound speed measurement.

### Embodiment 3

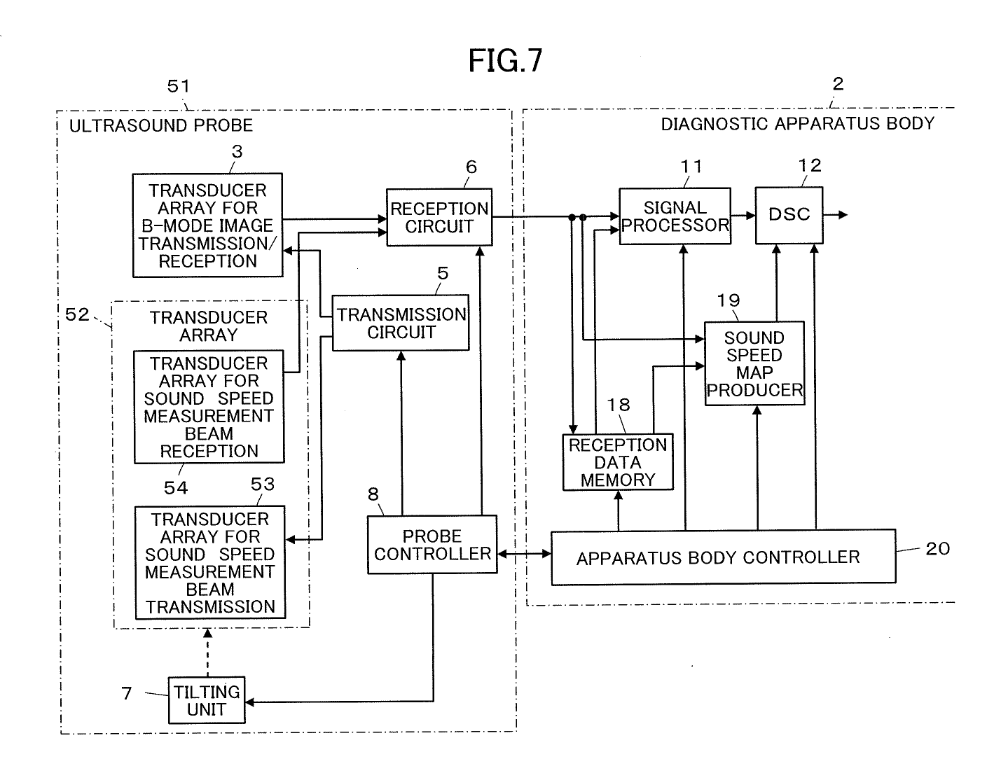

FIG. 7 shows the configuration of an ultrasound probe 51 according to Embodiment 3. The ultrasound probe 51 uses a transducer array 52 for sound speed measurement including a transducer array 53 for sound speed measurement beam transmission and a transducer array 54 for sound speed measurement beam reception, instead of the transducer array 42 for sound speed measurement beam transmission/reception in the ultrasound probe 41 of Embodiment 2 shown in FIG. 2. The transducer array 53 for sound speed measurement beam transmission is connected to the transmission circuit 5, and the transducer array 54 for sound speed measurement beam reception is connected to the reception circuit 6.

Even with this configuration, as in Embodiments 1 and 2, the tilt angle of the transducer array 52 for sound speed measurement is adjusted by the tilting unit 7, thereby reducing the influence of refraction of an ultrasonic beam from the abdominal wall P to perform accurate sound speed measurement.

In this case, a plurality of ultrasound transducers of the transducer array 53 for sound speed measurement beam transmission can be formed of piezoelectric ceramic represented by PZT (Pb (lead) zirconate titanate), while a plurality of ultrasound transducers of the transducer array 54 for sound speed measurement beam reception can be formed of a polymer piezoelectric device represented by PVDF (polyvinylidene difluoride).

If a polymer piezoelectric device is used for the ultrasound transducers of the transducer array 54 for sound speed measurement beam reception, it becomes possible to suppress the influence of side lobe to improve resolution in the depth direction and the direction perpendicular to the depth direction.

A low-frequency ultrasonic beam may be transmitted from the transducer array 53 for sound speed measurement beam transmission, and two or more harmonics may be received by the transducer array 54 for sound speed measurement beam reception, thereby enhancing main lobe and further reducing side lobe.

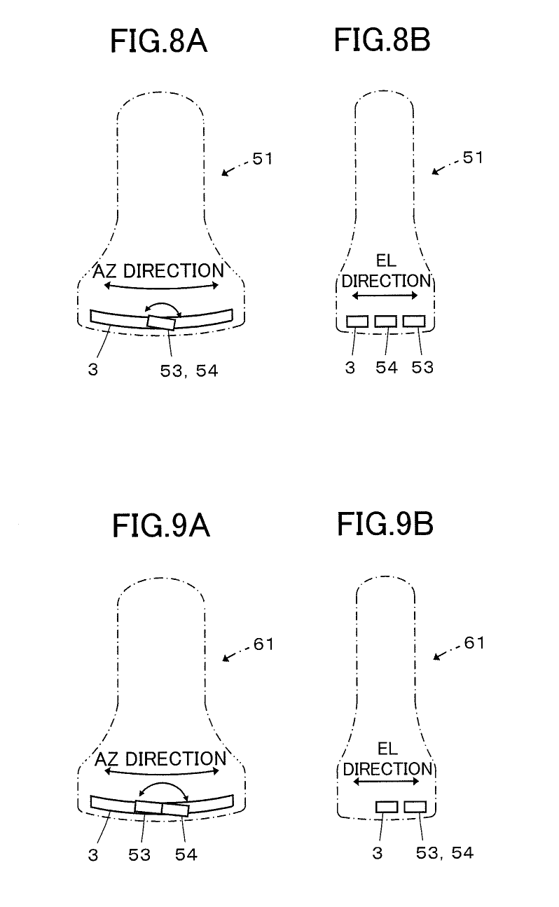

As shown in FIGS. 8A and 8B, the transducer array 53 for sound speed measurement beam transmission and the transducer array 54 for sound speed measurement beam reception may be sequentially disposed in parallel adjacently in the elevation direction of the transducer array 3 for B-mode image transmission/reception. The transducer array 53 for sound speed measurement beam transmission and the transducer array 54 for sound speed measurement beam reception can be configured to be tilted in the azimuth direction.

Like an ultrasound probe 61 shown in FIGS. 9A and 9B, the transducer array 53 for sound speed measurement beam transmission and the transducer array 54 for sound speed measurement beam reception may be integrally arranged as a single body so as to be adjacent to each other in the azimuth direction and may be disposed adjacently in the elevation direction of the transducer array 3 for B-mode image transmission/reception.

Though not shown, the transducer array 53 for sound speed measurement beam transmission and the transducer array 54 for sound speed measurement beam reception may be disposed adjacently in the azimuth direction of the transducer array 3 for B-mode image transmission/reception. The transducer array 53 for sound speed measurement beam transmission and the transducer array 54 for sound speed measurement beam reception may be laminated.

Although in the ultrasound probe 51 of Embodiment 3 shown in FIG. 7, the transducer array 3 for B-mode image transmission/reception and the transducer array 54 for sound speed measurement beam reception are commonly connected to the reception circuit 6, and the reception circuit 6 produces reception data for a B-mode image and reception data for sound speed measurement, an additional reception circuit for sound speed measured may be provided separately from the reception circuit 6, and the transducer array 54 for sound speed measurement beam reception may be connected to the reception circuit for sound speed measurement to produce reception data for sound speed measurement.

Similarly, an additional transmission circuit for sound speed measurement may be provided separately from the transmission circuit 5, and driving signals may be supplied from the transmission circuit for sound speed measurement to the transducer array 53 for sound speed measurement beam transmission to transmit ultrasonic beams for sound speed measurement.

Although in Embodiments 1 to 3 described above, reception data output from the reception circuit 6 is temporarily stored in the reception data memory 18, and the sound speed map producer 19 produces a sound speed map in the region of interest R using reception data stored in the reception data memory 18, the sound speed map producer 19 may directly receive reception data output from the reception circuit 6 to produce a sound speed map.

The reception data memory 18 stores not only reception data for sound speed measurement but also reception data for B-mode image production. For this reason, reception data for B-mode image production may be read from the reception data memory 18 as necessary under the control of the apparatus body controller 20, and a B-mode image may be generated by the image producer 23.

The connection of the ultrasound probe 1, 31, 41, 51, or 61 and the diagnostic apparatus body 2 in Embodiments 1 to 3 described above may be either wired connection or connection by wireless communication.

What is claimed is:

1. An ultrasound probe which transmits an ultrasonic beam toward a subject and receives an ultrasonic echo from the subject, the ultrasound probe comprising:
a first transducer array for a B-mode image which transmits an ultrasonic beam for a B-mode image;
a second transducer array for sound speed measurement which is tiltably provided and transmits an ultrasonic beam for sound speed measurement; and
a tilting unit which tilts the second transducer array for sound speed measurement in accordance with the angle of an abdominal wall of the subject.

2. The ultrasound probe according to claim 1, further comprising:
a transmission circuit which transmits an ultrasonic beam for a B-mode image from the first transducer array for a B-mode image and transmits an ultrasonic beam for sound speed measurement from the second transducer array for sound speed measurement; and
a reception circuit which processes a reception signal based on the ultrasonic echo from the subject to obtain reception data for a B-mode image and reception data for sound speed measurement.

3. The ultrasound probe according to claim 2,
wherein the reception circuit processes a reception signal output from the first transducer array for a B-mode image having received an ultrasonic echo of the ultrasonic beam for sound speed measurement from the subject to obtain reception data for sound speed measurement.

4. The ultrasound probe according to claim 2,
wherein the reception circuit processes a reception signal output from the second transducer array for sound speed measurement having received an ultrasonic echo of the ultrasonic beam for sound speed measurement from the subject to obtain reception data for sound speed measurement.

5. The ultrasound probe according to claim 4,
wherein the second transducer array for sound speed measurement includes an array for transmission and an array for reception, and
the reception circuit processes a reception signal output from the array for reception having received the ultrasonic echo of the ultrasonic beam for sound speed measurement transmitted from the array for transmission from the subject to obtain reception data for sound speed measurement.

6. The ultrasound probe according to claim 5,
wherein the reception circuit includes a reception circuit for a B-mode image and a reception circuit for sound speed measurement, and
a reception signal output from the array for reception having received the ultrasonic echo of the ultrasonic beam for sound speed measurement transmitted from the array for transmission from the subject is processed by the reception circuit for sound speed measurement and reception data for sound speed measurement is obtained.

7. An ultrasound diagnostic apparatus comprising:
the ultrasound probe according to claim 1;
an image producer which produces a B-mode image on the basis of reception data for a B-mode image obtained by the reception circuit;
an abdominal wall detector which detects the angle of an abdominal wall of a subject on a B-mode image produced by the image producer;
a controller which adjusts the tilt angle of the second transducer array for sound speed measurement by the tilting unit in accordance with the angle of the abdominal wall detected by the abdominal wall detector; and
a sound speed map producer which produces a sound speed map on the basis of reception data for sound speed measurement obtained by the reception circuit.

8. The ultrasound diagnostic apparatus according to claim 7,
wherein the controller adjusts the tilt angle of the second transducer array for sound speed measurement by the tilting unit so as to reduce the influence of refraction of the ultrasonic beam for sound speed measurement from the abdominal wall.
